(19) United States
(12) Patent Application Publication
(10) Pub. No.: US 2025/0290129 A1
(43) Pub. Date: Sep. 18, 2025

(54) Event-Based Sequencing of Nucleic Acids in Real Time

(71) Applicant: Stream Genomics, Inc., Carlsbad, CA (US)

(72) Inventors: Bojan Obradovic, Thaxted (GB); Tsu-Ju Fu, Poway, CA (US); Kay Klausing, San Diego, CA (US); Leopold Garnar-Wortzel, San Diego, CA (US)

(73) Assignee: Stream Genomics, Inc., Carlsbad, CA (US)

(21) Appl. No.: 18/803,758

(22) Filed: Aug. 13, 2024

Related U.S. Application Data
(60) Provisional application No. 63/532,606, filed on Aug. 14, 2023.

(51) Int. Cl. C12Q 1/6869; B01L 3/00; G06T 7/246; G16B 30/00

(52) U.S. Cl. CPC C12Q 1/6869; B01L 3/502; G06T 7/246; G16B 30/00; B01L 2200/026; B01L 2200/0647; B01L 2300/023; B01L 2300/025; G06T 2207/10016; G06T 2207/10056; G06T 2207/20081; G06T 2207/30004

(57) ABSTRACT

Real-time sequencing of single nucleic acids, using event-based detection for uniformly high signal-to-noise ratios and streamlined data outputs. Methods and devices for using temporal contrast pixel arrays (TCPA) to detect indicator molecules bound to targets, such as nucleic acids, enabling real-time, asynchronous sequencing. Instruments, consumable kits and devices, and point-of-care devices for performing these methods and displaying the results on mobile devices.

### CROSS-REFERENCE TO RELATED APPLICATIONS

This application claims the benefit of U.S. provisional application 63/532,606, filed Aug. 14, 2023, entitled Methods and Devices for Detecting Nucleic Acids in Real Time, the contents of which are incorporated herein in its entirety.

### TECHNICAL FIELD

The present invention relates to the field of biochemistry. More specifically, it relates to methods and devices for event-based imaging technology for real-time detection of chemical reactions, such as nucleic acid sequencing.

### SUMMARY OF INVENTION

The present invention provides event-based detection technology to streamline the detection of target molecules and to monitor binding events between molecules. The invention uses Temporal Contrast Pixel Arrays (TCPAs), an imaging technology inspired by the retinas of vertebrates, for the event-based detection. In many cases, a communications protocol termed Address Event Representation (AER) can be used to facilitate communication between the bio-inspired chips. This technology allows for independent detection of each incorporated nucleotide, measuring and reporting a signal in real time only when it changes. It also reduces the need for cycling through chemistries and wash steps, thereby improving speed and cost.

Speed is improved because the sequencing can proceed at the natural pace of the polymerase, rather than according to a predetermined cycle of steps that must be coordinated for multiple targets. Incorporation and removal of fluorescence is achieved on each molecule individually, as opposed to waiting for the incorporation to occur for all molecules at once. Imaging also occurs for each fluorophore independently, so there is no waiting for an average exposure time for multiple fluorophores.

Cost is reduced by the one-pot chemistry design, since all reagents required for the sequencing operation can be introduced at once. In contrast to conventional methods, which can use hundreds of milliliters of reagents, the one-pot design can reduce the volumes to tens of microliters. In some workflows, repeated wash steps become optional, thereby eliminating the waste associated with washing steps and with reintroducing additional reagents for subsequent cycles.

Disclosed more broadly herein, the present invention provides methods for using event-based detection like using the TCPA to detect changes in the emissions of indicator molecules that are bound to target molecules of interest. The indicator molecules can have a binding moiety to bind to a region of a target molecule and a detectable moiety for detection by the TCPA.

The present invention provides reagent cocktails of indicator molecules that can be used in the methods of the invention. Individual indicators can have binding moieties that bind to different regions of a target, as well as indicators having varying binding-moiety-specific detectable moieties, which can be selected to be surprisingly similar to others in the cocktail due to the subtle and efficient change-detection abilities enabled by the TCPA. Also provided are related kits of the cocktails with additional components useful in performing the methods, such as enzymes, reagents, and buffers.

The invention also provides instruments for performing the methods of the invention, having, for example, a TCPA, fluidic means for delivering and/or evacuating an indicator cocktail, means for communicating changes in image, and a system for collecting and analyzing change data. The instrument can be provided to the user in a “dry” configuration, so that the reagents required for the entirety of the sequencing operation can be contained in a separate consumable chamber with reagent solutions. In this dry configuration, without the need for reagents to be introduced to the chamber or evacuated from it, the instrument can be provided without liquid pumps and valves and their associated control hardware, thereby reducing the need for expensive testing of such components during manufacture. The instrument can also have sample chambers, excitation light sources, temperature controls, automated liquid handling systems, and a laboratory information management system (LIMS).

Other instruments of the invention include versions of the instruments in the form of a consumable device, which can incorporate single-use reagents. A particular version of the instrument takes the form of a point-of-care device that can perform some or all of the steps of the method and report the results on a mobile device.

### BRIEF DESCRIPTION OF DRAWINGS

FIG. 1 shows a sequencing reaction interface (1), such as the surface of a sequencing flow chamber, with target molecules of interest (2) located on the interface. In this version, the interface (1) can be imaged with a conventional detector (3) or an event-based detector (4) over multiple sequencing cycles (5a, 5b, 5c). Quantities of data are illustrative and depend on detector models.

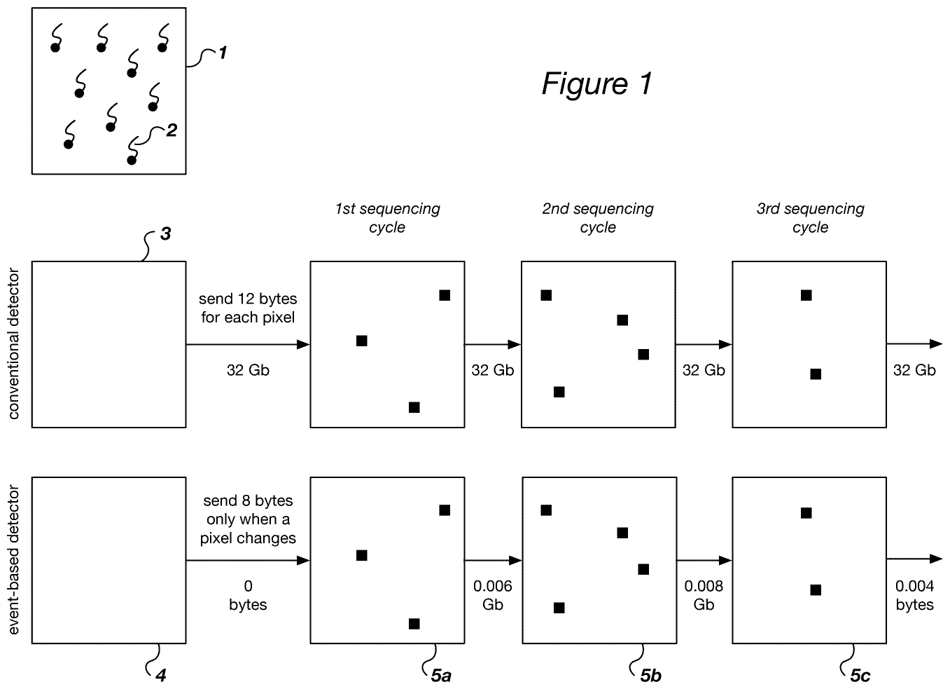

### DETAILED DESCRIPTION OF THE INVENTION

Current state-of-the-art DNA sequencing techniques either perform sequencing in a stepwise fashion—requiring separate steps for introducing reagents, incubation, washing, and imaging—or in real-time, using expensive sensors and time-consuming image analysis, which can require extensive computation, limits sequencing length, or introduces high error rates in the sequencing data. This invention provides a novel, cost-effective system for real-time sequencing by applying an innovative imaging technique.

The present invention provides event-based imaging for target molecule detection, such as in nucleic acid sequencing and in the monitoring of molecular binding events. The invention can use Temporal Contrast Pixel Arrays (TCPAs) for the event-based detection. A communications protocol termed Address Event Representation (AER) can be used to facilitate communication, such as spike information, between neuromorphic chips and image sensors to transmit pulses.

Inspired by the retinas of vertebrates, which only transmit information for change events occurring within the scene in view, and are not driven by an artificial timing mechanism, TCPA is an imaging technology where exposure measurements are locally initiated and carried out by individual pixels. Pixel circuits can be constructed so that pixels detect changes in brightness and independently communicate the differences in grayscale values, without relying on external timing signals. Thus, pixels in an array produce output only when stimulated, and the signal of each pixel can be read out independently.

Accordingly, the present invention provides event-based methods for using temporal contrast pixel arrays (TCPAs) to detect changes in the emissions of indicator molecules that are bound to target molecules of interest. By focusing on detecting only changes in signal that meet predefined criteria, this sharply reduces the amount of information that needs to be gathered, unlike conventional sequencing methods where the pixels of the entire sensor are read out in every cycle, whether or not each pixel in the array contains signal that is significant to the sequence.

The invention also provides methods for detecting the presence of target molecules of interest in samples. The methods can have the steps of: (a) providing a sample to a chamber; (b) attaching the sample to one or more surfaces; (c) performing the method of the invention for detecting changes (e.g., using a previous signal as a baseline) on the sample by providing at least a first indicator by (1) allowing the indicator to bind specifically to a first region of a target; (2) exciting the indicator to emit a detectable signal; and (3) reporting a change that is detected within a pixel or a group of pixels within an array of the TCPA; and (c) combining or analyzing the change information to detect the target. As a result, the method detects the presence of the target in the sample.

A particular embodiment of the method can have the steps of (a) providing the sample to a chamber; (b) performing the method of the invention on the sample by providing at least a first indicator by (1) allowing the indicator to bind specifically to a first region of a target; and providing at least a second or more kinds of indicator molecules that are not binding (or binding with significantly weaker affinity) to the first region, e.g., because each kind of indicator has a binding moiety and detectable moiety different from other kinds; (2) exciting the first indicator to emit a detectable signal in a fixed location while other unbound indicator molecules may be excited but are moving around in space, instead of stalling at a fixed location, so that they collectively become the background signal; and (3) reporting a change that is detected in a pixel or within a group of pixels within an array of the TCPA; (4) removing the detectable moiety while continuing to block another binding moiety from binding to the same region of the target; (c) performing step (b) with a different region of a target; and (d) combining or analyzing the change information to detect the target.

### Samples

The methods of the invention can be used to detect the presence of target molecules of interest in samples. When performing the method, the samples do not necessarily need to contain the target, but can merely be suspected of containing the target of interest, or can lack the target by design as in the case of a negative control sample. The samples can be artificially created, biological in origin, or taken from the environment. Environmental samples can be useful in environmental bio-surveillance, such as monitoring for certain viruses in institutional or public wastewater, and especially in public health preparation for future pandemics.

Biological samples can be or be derived from viruses, bacteria, microorganisms, plants, and animals, such as humans. The sample can be of a body fluid, such as a blood sample, cerebrospinal fluid, cells, or cell-free fractions isolated from a sample, or a tissue sample. The sample can also be from a culture medium that has been incubated with a sample collected from a patient suspected of having an infection caused by a pathogen, which serves as a target for detection.

### Targets

The target molecules to be detected can be any molecule that has a binding site suitable for binding by a moiety as described below. An example of a target is a molecule having epitopes that can be bound by an epitope-specific binding moiety, such as an antibody, antibody fragment, or aptamer. The target can also be a polymer that presents multiple regions for binding, such as a biological polymer. Examples of such polymers include nucleic acids, such as DNA and RNA, proteins, peptides, carbohydrates, and lipids. A target can be a component of a larger complex; for example, a target nucleic acid of interest can be part of a hybridized duplex, or even a complex including a hybridized DNA primer or a DNA polymerase.

Examples of DNA include nuclear or mitochondrial DNA, modified DNA, cell free DNA (cfDNA), circulating tumor DNA (ctDNA), synthetic DNA, and cDNA that is reverse transcribed from RNA. Examples of RNA include mRNA, rRNA, tRNA, miRNA (microRNA), snRNA (small nuclear RNA), siRNA (e.g., small interfering RNA, small inhibitory RNA, and synthetic inhibitory RNA), antisense RNA, circular RNA, circulating free RNA (cfRNA), circulating tumor RNA (ctRNA), long noncoding RNA, RNA containing unnatural bases, synthetic RNA, and modified RNA.

The target can also be a non-naturally occurring polymer, such as a nucleic acid having phosphate-sugar backbone analogs or nucleic acid analogs containing synthetic or unnatural (nonnaturally occurring) bases. Particular examples of non-naturally occurring nucleic acids include peptide nucleic acids (PNAs), locked nucleic acids (LNAs), glycol nucleic acids (GNAs), threose nucleic acids (TNAs), and hexitol nucleic acids (HNAs). Other examples of non-naturally occurring polymers that are similar to natural polypeptides or proteins, but include peptide analogs.

The target of interest can be used to detect the presence of a pathogen, a toxin, or the causative agent of a pathology, or detect a physical trait, such as a metabolic or genetic trait. An example is a sepsis-causing pathogen. When more than one pathogen may be present in a sample, such as 2, 5, 10, 15, 20, 25, 30, 35, 40, 50, 60, 70, 80, 90, 100, 120, 150, 200, 250, 300, 400, 500, or more targets, or targets in a range between any of the preceding numbers inclusive, the invention can provide methods for detecting panels of targets, such as targets associated with Gram-positive bacteria, Gram-negative bacteria, fungal pathogens (FP), viruses, multicellular parasites, and protozoa.

### —Immobilization and Continuous Flow

Targets can be bound or immobilized, directly or indirectly, to one or more surfaces of a substrate. Examples of indirect binding include capture by enzymes or capture probes, and subsequent immobilization on a surface. Template molecules can be anchored at multiple locations on the surface of the substrate and can be arranged in an ordered or random way.

The immobilization of a target on a substrate can be accomplished via direct hybridization to an immobilized capture probe that has a sequence that is complementary to at least a portion of the target. The complementary portion on the target can be native to the target or added in a separate step. The immobilization can be performed indirectly by hybridizing a capture probe (CP) that is biotinylated, for example. The target and biotinylated capture probe (CP) can then be captured on a streptavidin-coated surface, such as a glass surface inside a flow cell. In an embodiment, the target can be captured by a surface-bound enzyme or protein. For example, primer-template complexes can be immobilized by surface-bound DNA polymerases in a sequencing reaction. In another embodiment, the target can be captured by an enzyme or protein and subsequently immobilized on a surface via the enzyme or protein.

The method can further have the step of immobilizing at least part of the target on the substrate. Thus, the individual targets can be seeded onto the substrate, so the targets are sufficiently separated for optical resolution. This substrate can be transparent or opaque. It can be made into a flowcell or can be an open well, arranged individually or in the format of a multi-well plate, with a transparent bottom and/or sides.

The immobilization can be directly onto the surface, or can be near the surface, such as when the surface is coated or has an intermediate layer. In some embodiments, the immobilization can be within a three-dimensional matrix, so long as indicators that are bound to the target can be detected. The immobilization can be irreversible (e.g., covalently), reversible (e.g., by binding to an enzyme), or partially reversible or irreversible depending on the local environment or particular binding or releasing conditions, such as the presence of competitors or co-factors.

In some embodiments, the method can be performed as a continuous flow of indicators, where the target is not immobilized, but is located within a fluid that can flow in relation to a surface or substrate, for example within a flow cell. The fluid can be relatively viscous in order to move relatively slowly in relation to the surface, and so that the positions of individual targets can be identified and tracked over time in successive images despite not being immobilized. In other embodiments, the indicators can be provided to the sample chamber and allowed to diffuse freely.

### Indicator Molecules

The indicators used in the invention can have at least a binding moiety and a detectable moiety. The two moieties can be attached via a linker or other moiety.

### —Binding Moieties

The binding moiety can be capable of binding specifically to a region of the target. The specificity should be sufficient to bind to the intended region of the target while distinguishing between different possible binding sites that may be present at the region, depending on the nature of the target and the sample. The strength of the binding can vary, but should be sufficient so the binding of the appropriate indicator can be detected, and preferably significantly stronger than any nonspecific binding to undesired targets or by undesired binding moieties.

A binding moiety can be a modified nucleotide, which can have a general structure of F-L₁-(PO₃⁻)ₘ—O—(PYO⁻)-(ribose or deoxyribose)-B, where m is an integer of 1, 2, 3, 4, 5, 6, 7, 8, 9 or more; F is an optional label such as a fluorescent dye or protein; Y is O or S; L is a linker selected from alkyl, alkenyl, alkynyl, aryl, heteroaryl, heterocycle, polyethylene glycol, ester, amino, sulfonyl, or a combination thereof; and B is selected from adenine, cytosine, guanine, thymidine, uracil, or other base analog. F-L in the general structure can be replaced by a branched linker with multiple fluorescent dyes or proteins. A nucleotide can be a dNTP, such as fluorescently labeled nucleotides (dA, dC, dG, dT). In a particular embodiment of the invention, the indicator can be a labeled but unterminated nucleotide.

The binding moiety can also be an antigen-binding fragment (Fab), such as an antibody, or an aptamer. The binding moiety can also be an oligonucleotide with a known sequence designed to hybridize to a region of interest of the target. Such probes can be labeled with single or multiple detectable moieties, and repeated hybridization events can be used to determine the sequence or composition of the target. The binding event can encompass the binding of the binding moiety to one or more sites or epitopes on a target. In some cases, the binding moiety can comprise two or more binding elements, for example, two or more binding domains that bind to two or more separate epitopes of a target, so that a plurality of molecules (serving as the binding moiety) bind specifically to a plurality of epitopes (serving as the target). For example, a target may comprise a multimolecular complex of a polymerase and a nucleic acid template, where the complex can be bound by a binding moiety that has a complementary nucleotide.

The binding between indicator and target can be mediated by an oligonucleotide, a protein, an enzyme, an organic compound, a buffer solution, or a combination thereof.

In further embodiments, an indicator can have a binding moiety that binds to more than one binding site, for example a binding moiety that has a degenerate sequence that can bind to multiple target sequences. This version can be useful in microsequencing applications, where the presence or absence of a significant but relatively short nucleotide sequence is to be detected, rather than sequencing a run of individual nucleotides on the target.

### —Detectable Moieties

The detectable moiety can be capable of emitting a detectable signal. For example, the detectable moiety can be or have a fluorophore, which is a chemical that can absorb light and re-emit light, often at characteristic excitation and/or emission wavelengths. The light can be in the infrared, visible, or ultraviolet ranges. Examples of fluorophores include organic fluorescent dyes (e.g., coumarins, cyanines, fluoresceins, rhodamines, and their derivatives). Useful dyes are commercially available, including Alexa Fluors (Molecular Probes, Thermo Fisher Scientific), Atto fluorescent dyes and labels (ATTO-TEC GmbH), and the DY-series of dyes (Dyomics GmbH). Other fluorophores include biological fluorophores (e.g., green fluorescent protein (GFP) and phycoerythrin), acceptor-x-donor fluorophores, lanthanide fluorophores for time-resolved fluorescence (TRF), FRET fluorophore pairs, or quantum dots. Dyes can be differentiated by differences in photobleaching times. The detectable moiety can also be detectable by Raman scattering.

The detectable moieties can be differentiated from one another by various dimensions, such as excitation wavelength, emission wavelength and brightness, duration of emission, fluorescence lifetime, or other temporal properties. In yet other embodiments, the signal from different detectable moieties can be differentiated on the basis of differing reaction properties, such as characteristic incorporation times by a polymerase and a particular nucleotide.

For each of these dimensions, there can be 2, 3, 4, 5, 6, 7, 8, 9, 10, 11, 12, 13, 14, 15, 16, 18, 20, 25, 30, 35, 40, 45, 50, 60, 70, 80, 90, 100 or more different indicators, or of a range between any of the preceding numbers inclusive. Moreover, differences in one dimension can be combined with differences in one or more other dimensions to obtain combinatorial sets of different indicators to distinguish between greater numbers of binding moieties than by wavelength alone. A first dimension of differences can distinguish between a first functional set of moieties and a second dimension of differences between a second functional set of moieties. For example, four different wavelengths (e.g., red, yellow, green, and blue) can distinguish between binding at four different subunits of a coat protein, and different emission durations (e.g., short, medium, and long) can distinguish between three antigen types at each subunit. As other example, a first dimension of wavelength can provide a rapid readout of the presence of subunits of interest, and the emission durations can provide a more nuanced readout between individual antigen types.

In other embodiments, “dark” indicators can be provided that bind to a region of a target molecule, preventing binding of or competing with other indicators, but emitting no or less light than other indicators. For example, a ribosomal sequence can have relatively invariable regions that have less interest than more variable regions that are characteristic of a pathogenic species.

### Compositions

The invention provides compositions obtained by performing one or more steps of the method.

The invention also provides multiple indicator molecules, having different binding and/or detection properties, which can be provided as cocktails of multiple indicators. The cocktails can also be provided with components such as a protein, a quenching reagent, a molecular crowding agent (e.g., sucrose, polyethylene glycol (PEG), Ficoll®, dextran, or serum albumin), a reagent to reduce or enhance blinking, and a component to increase viscosity. Other miscellaneous components include detergents, reagents and enzymes, such as a polymerase and buffer. The components can be provided combined with the indicators or packaged separately.

### TIRF Illumination and Excitation

A useful means for illuminating or exciting the detectable moieties of the indicators is evanescent wave excitation, where the energy of an oscillating electric and/or magnetic field is concentrated in the vicinity of the source, which can be described as an evanescent wave field. Total Internal Reflection (TIR) occurs when a light beam travels within a medium with a higher index of refraction and meets the interface of a medium with a lower refractive index at an angle greater than a critical angle. The change in direction of the light propagating through the interface is described by Snell's law of refraction, nisin (θ₁)=n₂ sin (θ₂), where n₁ is the index of refraction of a medium/with a higher refractive index (e.g., a solid such as glass), n₂ is the index of refraction of a medium 2 with the lower refractive index (e.g., liquid or gas), θ₁, is the angle of the light in medium/towards the normal, and θ₂ is the angle of light in medium 2 towards the normal. By way of illustration, for a typical aqueous buffer, n₂ could be 1.33, and for glass, n₁ could be 1.52.

For angles of incidence where q₁>q_{c} and where θ_{c}=arcsin (n₂/n₁), the electric field component of the light penetrates through the interface into the second medium as an evanescent wave. The intensity of the wave decays exponentially with distance from the interface into the second medium with a characteristic penetration depth that depends on the wavelength and the angle of incidence of the light.

Total Internal Reflection Fluorescence (TIRF) microscopy takes advantage of the evanescent wave generated by TIR. TIRF can be implemented using a prism on one side of the substrate, known as prism-based TIRF, or through the objective lens, known as objective-based TIRF. In either case, the light beam is propagated through the denser medium (glass) at an angle greater than q_{c} and the total internal reflection is achieved at an interface with the lower index of refraction (substrate, liquid, or gas).

TIRF selectively excites fluorophores near the surface-typically within 100 nm, but generally not more than 1 mm-without significantly affecting fluorophores further away. The detectable moieties of the indicators can be detected during the time period that they are located or stationary within the evanescent wave field, the duration of which is described herein as “dwell time”.

This selective excitation differentiates between indicators bound to targets on the surface and those unbound in solution. For instance, TIRF can excite fluorescence in labeled nucleotides incorporated into a polynucleotide strand that are hybridized to a DNA template on a surface. Background can be reduced because fluorescent labels that are interacting with surface-bound template are excited, while labels in the solution remain unexcited. As a result, fluorescence from non-interacting, free-moving labels is not detected, thereby enhancing detection sensitivity.

The evanescent wave near the surface can also be achieved by propagating the beam through a medium of higher refractive index (layer I) deposited on top of the material of lower refractive index (layer II). The layer I is in contact with the sample and the reagents cocktail.

Layer I can be composed of a material that has a higher refractive index than that of layer II, for example TiO₂, ZnO, SiN, Nb₅O₅, Ta₂O₅, HfO₂, ZrO₂, Al₂O₃, TiO₂, SrTiO₃, diamond, SiC. The thickness of this layer I can be 10 nm up to 1 mm.

A grating can be etched within, or on top, or underneath layer I or in the layer II. The light incident upon the grating is at an angle equal to that of the first order of diffraction of the grating, then the light can be coupled into layer I (also known as the planar waveguide) and will propagate freely within layer I, but also projecting an evanescent wave on either side of layer I into layer II and into the cocktail liquid. There can be another grating some distance away from the first grating so that the light can propagate between the two gratings, whereby the light enters the planar waveguide by coupling in with grating 1 and exits the planar waveguide by coupling out of the waveguide with grating 2. This can be useful for monitoring the exiting light for its intensity or wavelength or any other property that may be desired. It is particularly useful to reflect the light after exiting grating 2 back on to grating 2 in order to couple it back into the planar waveguide and thus increase the illumination intensity.

The light can be coupled into layer I by means other than diffraction. For example, it can be focused into the side of layer I and thus directly launched in layer I. In another example, it can be coupled in via a prism in contact with layer II. In this case, layer II can be any material. Layer II can be a glass, a plastic, a metal, or a semiconductor, and it can be transparent or opaque.

Another method for creating the evanescent wave field in the aqueous cocktail medium is by propagating the light directly through the substrate, layer II, which can be achieved by focusing the light into the side of the substrate or by bringing it into contact with an optical fiber. In this method of creating an evanescent wave field, the substrate medium can be transparent and of higher refractive index than the aqueous cocktail solution. The substrate medium can be of a thickness of 100 nm, 200 nm, 500 nm, 1μ, 2μ, 5 μm, 10μ, 20 μm, 50 μm, 100 μm, 200μ, 500 μm, 1 mm, 2 mm, 3 mm, or any range thereof, inclusive.

### Temporal Contrast Pixel Arrays (TCPAs)

The fluorescent signal from indicators can be collected by an objective lens to form one or more images. The different wavelengths of fluorescence channels can be separated by interference filters and dichroic mirrors for detection as pixels in their corresponding arrays. In a four-color embodiment, for example, the image can be split and formed on four separate detection sensors, one for each fluorescence channel. The image can also be split and formed on four regions of the same sensor. In another embodiment, the signals can be collected by a multi-junction optoelectronic sensor. In one such sensor, for example, nanophotonic layers are stacked together onto each CMOS pixel to form a triple-junction photodiode that can distinguish between multiple wavelengths.

Temporal Contrast Pixel Arrays (TCPAs) can be used where exposure measurements are initiated by individual pixels only when they detect a change in the level of illumination within their field of view. Pixels in an array can be arranged in an orthogonal grid, a hexagonal pattern, or in a random or semirandom geometrical pattern; a TCPA may have regions of one arrangement and regions of another arrangement. Pixels respond to a variation in brightness and independently communicate new values to an output channel without relying on external timing signals. Each pixel can report 1, 2, 4, 8, 16, 32, 64, 128, 256, 512, 1024, 2048, 4096 or more bits of data.

A sensor useful in the invention can have a field of view of 0.1, 0.2, 0.5, 1, 2, 5, 7, 10, 12, 15, 17, 20, 22, 25, 30, 35, 40, 45, 50, 60, 70, 80, 90, 100, 150, 200, 300, 400, 500, 750, or 1000 or more megapixels (Mpx). In conventional image sensors, all pixels report brightness values in each frame of exposure, resulting in an enormous amount of data, most of which is redundant from image to subsequent image. By contrast, a TCPA can report only the data for pixels that have changed their brightness and only when such an event happens. This can lead to data compression of up to 100-, 200-, 300-, 400-, 500-, 700, or 1000-fold for scenes that are relatively static, as it is the case for an array of DNA molecules being sequenced. Thus, the amount of imaging data that is generated can be much less and easier to process for sequencing.

Using event-based detection, the output from a pixel can be reported as a change if there is a sufficient difference in a preselected numerical parameter. The change can be any increase or decrease in a parameter of the intensity or time domain, or can be based on a method, formula, or algorithm that receives such parameters as inputs. The time of the change, as well as other temporal properties such as duration or decay time parameters, can be recorded and associated with the pixel.

The detectable or reportable change can be a difference in brightness (intensity), emission wavelength, or polarization (linear, circular, or elliptical). The change can be based on the spiking properties of the brightness or intensity, such as rate of increase, duration, or decay profile, such as measured by half-life. The change in duration can be characterized by the time interval between the time points when the intensity reaches 10%, 20%, 25%, 30%, 40%, 50%, 60%, 70%, 75%, 80%, or 90% of the maximum intensity of the rising or falling signal.

The method for reporting a change can consider changes detected by adjacent pixels or pixels a predefined distance away. An aperture of a set of adjacent pixels can be analyzed, for example a central pixel and 1, 2, 3, 4, 5, 6, 7, 8, 9, 10, 11, 12, 14, 15, 16, 18, 20, 22, 25, 30, 35, 40, 45, or 50 or more adjacent pixels, or a group of preferably contiguous nearby pixels, such as within 1, \sqrt{2}, \sqrt{3}, 2, 3, 4, 5 or more pixels' radius away from the central pixel, whether measured radially, orthogonally (along perpendicular coordinates), or hexagonally, as appropriate to the layout of the pixels on the array grid. In some cases, signals from selected nonadjacent pixels can be analyzed to increase computational efficiency. In some cases it is possible to employ super-resolution techniques such as stochastic optical reconstruction microscopy (STORM), photo-activated localization microscopy (PALM), or resolution enhancement by sequential imaging (RESI) to increase resolution so that multiple objects can be detected in each pixel.

The method can also receive information from a moving window of the intensity or time domain.

The method can optionally be adjusted for local or global changes (such as drift) in the fluorescence detected in the array of pixels. Other factors can be incorporated into the method for reporting a change, such as the speed at which the signal appears, signal response, photobleaching, blinking, death numbers and survival times.

The change can be of a quantitative threshold of greater than 10, 15, 20, 25, 30, 35, 40, 45, 50, 55, 60, 65, 70, 75, 80, 90, 100, 120, 130, 140, 150, 200, 250, 300, 250, 400, 500, 600, 700, 800, 900, 1000, or more %, or any range between any of the preceding numbers inclusive. The change can also be based on a change or difference in value or sign of the first, second, third, or higher order derivative of the preceding parameters.

### —Pulse Width Modulation & Pulse Frequency Modulation

In a particular embodiment, event-based pixels can be configured in pulse width modulation (PWM) or in pulse frequency modulation (PFM) configurations. The TCPA sensor can use pulse-width modulation to report intensity values. This is a time-based detection mode that relies on the difference in time between a starting and ending trigger value to report the brightness. This enables the transformation of intensity signals into timing events on the pixel level, with each pixel independently reporting at a good signal-to-noise ratio (SNR) and with a high dynamic range. It is therefore useful to achieve a required threshold signal on each pixel, for example, in the case of nucleic acid sequencing, within the time window allowed by the incorporation time of the next nucleotide. In other words, this signal embodiment uses incorporating a labeled nucleotide to reside long enough in the sequencing complex for it to be detected. This allows us to capture the necessary fluorescence events for real-time sequencing of single DNA molecules.

In PWM, incident light generates a charge on the pixel as in a standard CMOS camera, but this output is then connected to the OP-AMP comparator, which toggles the state when this input value goes beyond some reference value. An external signal can be used to reset the sense node, and the pixel operates as a timer. The output of the comparator stays high for the duration of the pulse, thus the signal on each pixel is converted to the time of the pulse.

If instead of the external reset signal, the output of the comparator is used as a reset signal, the pixel becomes an oscillator that generates pulses on the output node, at a frequency related to the photocurrent (PFM).

An array of fully autonomous pixels that combine a change detector and a conditional exposure measurement device can therefore be constructed, and can be described as Temporal Contrast Pixel Arrays (TCPAs). For each pixel, the change detector can individually and asynchronously initiate the measurement of a new exposure after a brightness change of a predefined magnitude threshold or change beyond a defined range has been detected by the pixel. The pixel does not rely on external timing signal and independently reports output only when it has a new numerical value (“gray level”) to communicate. Pixels that are not stimulated or are insufficiently stimulated do not produce output.

The TCPA can be a complementary metal-oxide-semiconductor (CMOS), a Charge-Coupled Device (CCD), a scientific Complementary Metal-Oxide-Semiconductor (sCMOS), an electron-multiplying Charge-Coupled Device (emCCD), an intensified Charge-Coupled Device (iCCD) or a Micro Channel Plate detector (MCP). These technologies can be combined, for example, emCCD and iCCD to establish an emICCD. The TCPA can have photomultiplier tubes (PMT), Silicon Photomultipliers (SiPMs), Avalanche Photodiodes (APD), Single-Photon Avalanche Diodes (SPAD), or Quantum Dot Detectors (QDD).

### Instruments & Devices

The invention also provides an apparatus having: a TCPA; fluidic means for delivering and optionally evacuating an indicator cocktail or a wash solution; means for communicating changes in image; and a system for collecting and analyzing change data.

An embodiment of the apparatus of the invention takes the form of a consumable device that has a subset of components of the apparatus to perform the method, and suitable for a single use.

The apparatus can have means for controlling the temperature of the sample and/or the cocktail. For example, the temperature control means can be a CMOS device.

The apparatus can have an excitation light source, with an optional means for providing power to the light source. Examples of light sources include lasers, LEDs, or other light sources (e.g., tungsten light), with or without wavelength filters. In a particular embodiment, its baseline performance (photon transfer curve, optical or modulation transfer function) can be tested using a conventional scientific-grade CMOS camera equipped with a high numerical aperture (NA) microscope objective.

The TCPA can have a wave-guide surface or a coating where a layer of higher refractive index is deposited on a substrate of lower refractive index. Light waves launched into this high-refractive index layer are trapped inside, undergoing total internal reflection on the interface between the high-refractive-index medium and the low-refractive-index medium. Examples of such structures include layers of TiO₂, ZnO, SiN, Nb₅O₅, Ta₂O₅, HfO₂, ZrO₂, Al₂O₃, TiO₂, SrTiO₃, diamond, SiC, or similar material with refractive indices in the range of about 1.6 to 3. These materials can be deposited as a thin film with thicknesses varying from about 10 nm, 20 nm, 50 nm, 100 nm, 200 nm, 500 nm to 1 μm.

Such an apparatus can further have a sample chamber, which can hold single or multiple samples such as a 96-well, 384-well plate, 1536-well, or a specially designed plate (e.g., different well numbers and/or well size), or a PCR plate.

The sample chamber can be a flow cell in some embodiments and can comprise multiple lanes. There can be 1, 2, 4, 6, 8, 16, 24, 32, 64, or more sequencing lanes, for example. Light signals from events within the flow cell (optionally collected by a microscope) can be directed to separate collection channels. A target molecule, complex or complex component can be immobilized on the substrate surface directly or via a surface-bound molecule, e.g., a protein (such as streptavidin), an enzyme, a DNA nanostructure, a bifunctional linker, or a combination thereof.

In a particular consumable sample chamber, the chamber can be provided as a blister pack, for example where a storage buffer or a cocktail of indicators is preloaded. Accordingly, the invention encompasses a device that contains a sample and an indicator cocktail in the sample chamber. The method can also have a step of flushing the sample chamber.

The chamber can have means, such as a fluidic port, for receiving and optionally evacuating the sample. The fluidic port can also receive and/or evacuate the indicator cocktail or wash solution of the invention.

The apparatus can be provided with an automated liquid handler.

The device can also have means, such as a port, for communicating changes in an image from the TCPA to a system for data collection and analysis. The device can also have a means for supplying power to the TCPA.

In some embodiments, the apparatus can have a LIMS system.

The algorithm of the invention can transmit raw or partially processed output to a mobile device.

### Sequencing

The invention provides direct sequencing of single, individual DNA molecules, eliminating the need for template molecule amplification. The target can be an unamplified molecule, which is to say a molecule that has not been amplified or clonally copied prior to performing the detection or sequencing methods of the invention on the target molecule. The unamplified target can also be described as an individual or single molecule in the sense that the methods can be performed on a target molecule in a sample, rather than starting with a target molecule, amplifying the target, such as with PCR or isothermal amplification, before providing the sample with the target to a chamber. Direct RNA Sequencing (DRS) means performing sequencing on RNA molecules directly without amplification. Poly-A-tailed RNA targets can be captured and enriched by immobilized poly-dT sequences, such as a biotinylated poly-dT oligo, which can be manipulated with conventional methods. Other techniques can be used to manipulate target nucleic acids using a molecular handle, for example a chemical or physical moiety that has a cleavable disulfide bond. Sequencing can also be performed on cDNA molecules that are generated in situ from captured RNAs inside sequencing chambers, for example using species-specific or target-specific sequencing primers to capture their respective RNA targets. In some embodiments, the target molecule can be a polypeptide.

The method of the invention can involve binding indicators to regions of a target polymer in a stepwise manner, for example nucleotides in a nucleic acid or amino acids in a polypeptide. The binding can also be nonsequential, such as different epitopes on an antigen. The steps can be performed more than once at the same region of a target molecule, or by binding a plurality of moieties independently or asynchronously to one or more targets. By determining the properties of the target molecule at different regions, the sequencing of the target is achieved. The sequencing process is not required to be synchronized, and individual template molecules can be at different sequencing cycles. Thus, an embodiment of the sequencing chemistry can be described as continuous, rather than a cycle-based or pause-and-go reaction.

In a particular embodiment applied to a use for nucleic acid sequencing, a sample is prepared by purifying and isolating DNA or RNA, such as mRNA or rRNA. The nucleic acid can be fragmented, for example by physical means or by using restriction enzymes. The nucleic acid can be end-capped, for example by using a faustovirus or vaccinia capping enzyme (optionally with an mRNA cap 2′-O-methyltransferase) to add a 5′-m7G cap. Such caps or various hairpin (stem-loop) structures can be attached to one or more ends of a target molecule, such as a linear duplex nucleic acid. A strand of a duplex nucleic acid can serve as a template molecule.

In one embodiment, the template molecules can be immobilized on a surface, for example, the upper side of a flow-cell. The template can be part of a sequencing complex having components that include one or more of a primer, a template, and a polymerase, any of which can be surrounded by a solution that contains base-specific, fluorescently labeled nucleotides (e.g., dA-dye1, dT-dye2, dG-dye3, and dC-dye4). Each nucleotide can have a base-specific fluorescent probe attached on its gamma (terminal) 5′-phosphate. In another embodiment, each nucleotide can have a base-specific fluorescent probe attached on the 3′-end of the sugar via an ester bond. In a particular embodiment of SBS, each nucleotide can have a base-specific fluorescent probe that is attached to its base by a linker having a cleavable bond, for example, a disulfide bond (—S—S—).

The incorporation process in the sequencing method of the invention can be described as a series of several steps, without being bound to a particular order, depending on the components selected for a particular application. In one sequence, a template molecule is immobilized in the vicinity of an evanescent wave field. The template may be immobilized by binding to a polymerase, for example, which may also bind a primer nucleic acid. A labeled nucleotide in solution can come in close proximity to the template molecule and the polymerase, so that the labeled nucleotide binds to a catalytic incorporation pocket of the polymerase. The duration of the binding of the labeled nucleotide in the catalytic pocket can be described as a “residence time”, which is typically a subset of the “dwell time”. Typically, in the process, the labeled nucleotide pair-bonds (i.e. canonical Watson-Crick base pairing) with the nucleotide at the complementary position of the template strand to ensure binding of the correct complementary labeled nucleotide. As a result, the label enters the evanescent wave field and remains there for the duration of a “dwell time”, resulting in an increase in detectable fluorescence near the flow cell surface. The 5′-end of the labeled nucleotide becomes incorporated into the nascent strand by the polymerase. If the polymerase has an esterase activity, a subsequent cleavage of the ester bond by the polymerase at the 3′-end of nascent strand frees the fluorescent label into the surrounding solution and also provides a free 3′-end OH group that is ready for the next round of nucleotide incorporation. The ester bond can also be cleaved by an appropriate chemical or enzyme or physical condition (such as a change in pH) to release the label molecule from the sequencing complex. Thus, label can then be removed by the polymerase itself during strand extension, and no residual fluorescence remains within the evanescent wave field. The release of the label and movement beyond the evanescent wave field can be described as the conclusion of its dwell time, although the pattern of diminution and eventual cessation of detectable signal is marked by quantitative characteristics that become useful for characterizing the identity label and its physical context. In turn, another labeled-nucleotide incorporation event can follow, resulting in another increase in detectable fluorescence.

Additionally, in many embodiments, only those nucleotides with bases that are actually incorporated can contribute to the signal, since incorrectly bound or mismatched bases do not remain in the enzyme pocket (binding site or active site) long enough to be measured or its signal may appear on camera distinguishably shorter in time. Event-based detectors or cameras, such as TCPAs, then enable the independent detection of each incorporated nucleotide, for example of a growing nascent strand on a target nucleic acid in real time.

It can be useful to optimize the process of nucleotide incorporation by matching the incorporation speed to the imaging time requirement. DNA-dependent DNA polymerases generally join nucleotides in a chain at a few hundred nucleotides per second. In order to slow this process, a naturally occurring DNA polymerase can be obtained or existing polymerase can be modified to have an incorporation time greater than 10 ms (an incorporation rate of less than 100 nt/sec), or even greater, such as 20, 50, 100, 200, 500 or 1000 ms. Given the detection constraints of measuring the signal of a fluorescent dye, a reverse transcriptase that has a much lower reaction rate, e.g., with an estimated speed of 5 nt/see or 25 nt/see (200 ms or 40 ms per nucleotide incorporation rate), can also be used.

Useful polymerases include a DNA-dependent polymerase, an RNA-dependent polymerase, or a protein that has DNA-dependent polymerase or an RNA-dependent polymerase activity. In a sequencing reaction, one or more polymerases can be selected from DNA polymerase I, Klenow fragment of DNA polymerase I, DNA polymerase III, 9°N polymerase, B103 polymerase, Bst polymerase, rBst DNA polymerase, Bsu polymerase, GA-1 polymerase, KOD polymerase, N29 DNA polymerase, M2 polymerase, Pfu DNA polymerase, phi29 polymerase, PRD1 polymerase, SP6 RNA polymerase, a reverse transcriptase (e.g., AMV reverse transcriptase, MMLV reverse transcriptase, ProtoScript® II reverse transcriptase, and SuperScript® III reverse transcriptase), T3 DNA polymerase, T3 RNA polymerase, T4 polymerase, T5 polymerase, T7 DNA polymerase, T7 RNA polymerase, KlenTaq® polymerase, Taq polymerase, TopTaq polymerase, Deep Vent® DNA polymerase, Vent® DNA polymerase, Vent® (exo) polymerase, or variants or derivatives thereof.

The sequencing method can result in read lengths of 20, 50, 75, 100, 125, 150, 200, 300, 400, 500, 600, 750, 1000, 2k, 5k, 10k, 20k, 50k, 75k, 100k, 150k, or 200k bases or more, or read lengths between a range of any of the preceding lengths inclusive.

In summary, the invention provides methods for sequencing nucleic acids by detecting the presence of multiple regions (i.e. bases at individual nucleotide positions) of a target molecule. To start, a sample containing the target nucleic acid is typically provided to a sample chamber. Multiple indicator molecules can be provided, such as different nucleotides that have been modified with different fluorescent labels, so that one (or more) of the indicators binds specifically to one nucleotide position (“region”) of the target. When the indicator is bound to a polymerase, which is also bound to the target and a primer to form a sequencing complex, the indicator is within the evanescent wave field. Typically the indicator is excited to emit a detectable signal, and the signal is detected as a change by a pixel or group of pixels within an array of a TCPA. The detectable moiety can then be removed from the vicinity of the evanescent wave field. Around the same time, the nucleotide is incorporated into the nascent strand, thereby blocking other indicators (e.g., other detectably labeled nucleotides) from binding to the same, corresponding region of the target. The steps are repeated for a next position (region) on the target, further extending the nascent strand. The information obtained from the changes detected by the TCPA is then used to determine the sequence of the target nucleic acid.

### —Real-Time Detection

The detection can be in real time, and the time of detection can be recorded. Real-time chemistry can be achieved by removal of the fluorescence by the polymerase but also by constructing a donor-acceptor FRET pair between the enzyme and the dNTP. If the donor is located on the enzyme, the acceptor on the dNTP will only emit light when in close proximity to the enzyme.

Unlike conventional stepwise SBS (sequencing by synthesis), real-time imaging of each complex can monitor each primer extension step, enabling asynchronous, real-time sequencing by synthesis (rtSBS). This can be carried out by splitting imaging signals into a plurality of separate channels of sensors dedicated to individual bases by the corresponding wavelength of its label. For example, selected wavelengths or wavelength ranges can be directed to different temporal contrast pixel arrays (TCPAs), such as by using sets of dichroic mirrors. In some embodiments, a multi-wavelength image signal can be split into four dedicated wavelength channels (e.g., one for the emission wavelength associated with each nucleotide), eliminating crosstalk between channels, or where some crosstalk occurs, but is minimized or resolved by downstream processing. Another embodiment can use fewer channels (e.g., a dichroic mirror to separate each of three separate wavelengths, where the fourth wavelength is allowed to pass through). In other embodiments, a multi-wavelength signal can be separated into fewer than four channels, for example where a wavelength is shared by two or more nucleotides, but the signal for each nucleotide is further differentiated by a different detectable property, such as relative brightness/darkness, fluorescence lifetime or decay times, or polarization properties.

Asynchronous signal changes can arise from the incorporation of labeled nucleotides in sequencing complexes that are corelated with the sequences of their corresponding templates.

This can allow tracking of multiple, individual signal-changing events and tagging the events with an identifier of a specific coordinate in the field-of-view. This multiplexing capability does not require physical compartmentalization for each single template.

### —Detection of Features Beyond Primary Sequence

The stream of data obtained from the TCPA is highly efficient, but is also highly sensitive to numerical parameters and patterns, enabling the interpretation of output to go beyond identification of the conventional four nucleotides of the primary sequence of a polynucleotide. For example, depending on the polymerase used, labeled nucleotides have been observed to have a longer dwell time at methylated template positions than at corresponding unmethylated positions, which results in a significant difference in the detectable change. Other examples of chemical modifications to DNA and RNA include methylations, such as N°-methyladenosine (m⁶A), N7-methylguanosine (m⁷G), and 5-hydroxymethylcytidine (5hmC). Additional features in the target can be detected in nucleic acids, such as other epigenetic modifications, nucleotides containing pseudouridine (Ψ), as well as non-canonical base-pairings, secondary structures, and binding by proteins and other factors.

The detection of non-primary features can be extended by the invention to modifications to polypeptides and other natural or artificial polymers. Polypeptides, for example, can have secondary structures of interest, which may depend on the treatment of the sample, as well as an array of post-translational modifications. As discussed below, detection of such features can be further enabled and optimized by machine learning on training sets of output from known targets.

### —Signal-to-Noise Ratio

The event-based detection technique, originally developed for low light applications, is ideally suited for sequencing of nucleic acids because the intensity of most pixels in an image do not vary in intensity unless a label undergoes a change; the use of such a strategy also leads to high sensitivity and broad dynamic range. The event-based camera operates independently on each pixel, collecting data asynchronously when triggered by changes in intensity. Thus, signals from individual DNA templates can be recorded when the correct fluorescent labels are held long enough in the sequencing complex (consisting of a polymerase, a template, and a primer).

If the timespan or duration of enzymatic incorporation is matched to the signal-to-noise ratio (SNR) requirements of the detection method, real-time sequencing from single molecules is achievable. The method offers the advantage of reducing data size compared to full-frame readout methods, since only pixels experiencing a change of intensity are recorded.

In order to maximize the SNR, distinct fluorophores, which are distinguished by their properties, such as absorption and emission spectra, can be used to label certain kinds of nucleotide (e.g., one or more of dA, dT, dG, and dC). The fluorophores can be selected in combination with dichroic mirrors and/or interference filters. One consideration for selection can be to avoid spectral crosstalk or to minimize or manage the effect of such crosstalk.

This unique characteristic of maintaining a consistent SNR in event-based sensor circuits is extremely beneficial in fluorescence microscopy. This is especially true when the brightness of all fluorescent objects within the viewing field is not uniform. In traditional fluorescence microscopy, the exposure setting is usually calibrated based on the average brightness of the objects in the field. This often leads to inconsistencies in the quality of the images captured for particular objects, as some objects may be underexposed or overexposed depending on their relative individual brightness. On the other hand, when event-based imaging is used in fluorescence microscopy, the exposure time for each object is determined by the brightness of the object itself. This means that every fluorescent object in the view, regardless of its individual brightness, can be adjusted to achieve the same signal-to-noise ratio. This results in uniformly high-quality images, enhancing the details captured from each object in the field of view and ultimately yielding better data.

To further improve SNR, fluorescent signals can be restricted to surface-bound events using evanescent-field excitation. For example, when a labeled nucleotide is associated with a surface-bound template-primer-enzyme complex in the evanescent field, a small group of pixels on the corresponding sensor detects an event. Brief “wrong” nucleotide events can be ignored due to their far shorter interaction with the enzyme complex, and the vast majority of circulating, flowing, or diffusing fluorophores are not excited as they are outside of the evanescent field. The event time is recorded, and a base-call can be made on the channel detecting the event based on pre-defined criteria, or for example if it matches the expected signal characteristics, such as those defined by a Machine Learning (ML)-trained algorithm.

To coordinate the reaction speed of a DNA polymerase with the imaging requirements of fluorescent signals from single fluorophores (such as tens to hundreds of milliseconds to record a signal), the incorporation speed of the enzyme can be tuned to the sensor's requirements through manipulation of temperature, viscosity, pH, concentration, salt composition or concentration, buffer, or modifications to participating dNTPs. The window of time for measuring a sequencing event can be 0.5, 1, 2, 5, 10, 15, 20, 25, 30, 35, 40, 45, 50, 55, 60, 65, 70, 75, 80, 85, 90, 100, 110, 120, 130, 140, 150, 175, 200, 300, 400, 500, 600, 750, or 1000 milliseconds, or any range thereof, inclusive.

An advantage of real-time sequencing is that the sequencing operation is not limited by the slowest reaction step (often the time required for comprehensive imaging); rather the steps for each of the individual molecules can proceed at their own natural pace. Instead, the steps of a sequencing process in a real-time method do not need to be synchronized in any way, such as by an external clock that sets the starting times and duration for individual sequencing steps. For example, when a new signal appears in a channel, it can mark the beginning of a new incorporation event, as well as the end of the previous incorporation event. Under this timing scheme, there is no overlap between consecutive sequencing events, and the length and signal-shape/pattern of each event can be meaningful, both quantitatively (as in the length of signals detected from a homopolymer) and qualitatively (as in assignments of individual sequences of a difficult-to-sequence region).

Another advantage of the real-time sequencing method is the cocktail of reagents does not need to be added with each cycle as in conventional stepped methods, and can be added only once, or replenished as needed. This leads to savings on reaction time and reagent costs for real-time sequencing-by-synthesis (rtSBS). In another embodiment, the cocktail can be provided as a flow of reagents to provide indicators to the immediate location of immobilized targets.

In an embodiment, the event-based imaging technology can also be applied in stepwise sequencing schemes, requiring less data and computational power to process.

In another embodiment, the invention can be applied to detect the population frequency of sequence variants appearing in samples. Oligonucleotides, polynucleotides, or other probes (e.g., molecular beacons) with different labels can be designed to probe different variants of a gene. For example, the copy number of a mutated gene that causes or is associated with cancer may be very small compared to the copy number of normal or unmutated genes. Due to the highly sensitive readout of individual molecules provided by the present invention, minute fractions of mutated sequences can be detected, often without requiring a gene amplification step, thereby increasing the reliability of disease staging.

### Molecular Binding Assays

By combining the technologies of single-molecule detection and event-based imaging, the present invention also provides a microscopic approach to study binding assays, where binding and detachment events can be monitored in real time, even at the single-molecule level.

For example, the number of binding events between an antibody (or portion of an antibody, such as a Fab) and its target can be measured. The measurement can be within a fixed or predetermined window of time, or at different concentrations of antibody or target, to provide quantitative results. This enables the derivation of the binding strength or binding constant, and other properties of such binding events.

Other examples of binding include between two or more of a molecule selected from the group consisting of a chemical moiety, an organic molecule, a nucleotide, an oligonucleotide, a polynucleotide, a duplex DNA, a peptide, a protein (e.g., an enzyme, a nucleic-acid-binding protein, or a restriction enzyme), an aptamer, a hormone, a fatty acid, a lipid, a sugar, a simple or complex carbohydrate, a lectin, or a derivative or analog thereof.

### Algorithms and Machine Learning

The invention provides computer-performable algorithms for performing the analysis of the methods herein.

In some embodiments at least one of the steps can be stored or performed in cloud-based memory. A cloud system can provide on-demand computing resources and services, such as data storage and computing power, for user access from multiple remote locations and resources that can be distributed over multiple locations, often allocated dynamically and provisioned elastically.

In other embodiments, one or more steps can be stored or performed on a mobile electronic device or portable memory storage unit.

The invention also provides computer hardware and software that are capable using artificial intelligence (AI), such as by performing machine learning (ML) on training data sets. The training data can include TCPA output data from nucleic acids having known sequences, for example to translate data from actual samples into sequencing data with reduced ambiguity and improved accuracy. The algorithm can differentiate true incorporations compared to stochastic events arising from stray nucleotides and background. The algorithm can also distinguish homopolymers (repetitive stretches of the same base) and other difficult regions (e.g., GC-rich sections). The training sets can also include data from nucleic acids having known epigenetic modifications (such as methylation), non-canonical base-pairings, and secondary structures in order to improve the interpretation of TCPA output. AI can also be applied to review initial base calls, to flag close or borderline output for closer review, and to interpret difficult, duplicative, or conflicting regions, leading to reduced error rates.

In another example, an algorithm can be useful for identifying pathogens, for example sepsis-causing pathogens. Other uses include the diagnosis and treatment of cancer and rare diseases, and population genomics.

### EXAMPLES

### Example 1: Point-of-Care Device for Sepsis

Sepsis is an infection of the bloodstream and is a life-threatening medical condition that requires immediate attention to identify the infectious pathogen. It can cause extensive inflammation to tissues, organs, and throughout the body that can rapidly lead to tissue damage, organ failure, and even death. Bacterial infections are one of the most common causes for sepsis, with the most common causative bacteria include Staphylococcus aureus, Escherichia coli, Klebsiella pneumoniae, Streptococcus pneumoniae, and other Streptococcus spp. Fungal, parasitic, and viral infections can also be agents in sepsis.

According to a 2020 report published by the World Health Organization (WHO), sepsis (or septicaemia) caused the deaths of 11 million people annually, accounting for approximately 20% of all deaths globally. In the U.S., the total cost of sepsis hospital care for patients was $38 billion in 2017, with costs increasing by 8.8% annually.

However, conventional blood-culture approaches can take days to obtain results. Current molecular diagnostics for sepsis use oligonucleotide probes and nucleic-acid hybridization for detection means, but can require tedious sample prep. Thus, there is a need for a reliable test that is affordable and rapid, such as an hour from sample to result, at an affordable cost.

The invention also provides a high-speed sequencing platform that reduces the sample-to-result time for pathogen identification from days to hours, and can help determine the appropriate therapy, monitor patient responses to that therapy, and assess the patient's prognosis. The invention can also monitor the presence of pathogens for bio-surveillance of healthcare settings, public areas for potential exposure, and the environment.

The invention provides a point-of-care device for performing the steps of the method of the invention and displaying the results on a mobile electronic device. The point-of-care device can be used for diagnostic purposes. In an embodiment, four sensors with 2 million pixels each are integrated in an instrument with a microscope objective. A single field of view is illuminated by four lasers and real-time signal is collected on four sensors. This device obtains 100 Mb of genomic sequence data in 1 minute.

In one embodiment of the device, a first USB dongle plugs into a benchtop instrument and a second USB dongle plugs into a mobile device, such as a phone. One or both dongles can perform data analysis in the method of the invention.

The rapid sepsis test, which can be completed in about 60 minutes, begins with sample collection and extraction from blood to collect potential pathogens, then cell lysis to obtain rRNA. The rRNA is hybridized to capture probes to form a template for sequencing. No amplification of the template is required. The instrument performs real-time sequencing of multiple single molecules simultaneously, and yields base calls, which are assembled and annotated via cloud computing, resulting in the generation of an analysis report, including clinically actionable results. This test bypasses laborious and uncertain culturing tests, and time-consuming cycle-based sequencing and/or PCR methods, such as real-time PCR (rtPCR).

### Example 2: Whole Genome Sequencing

Whole human genome sequencing is performed by an instrument with four large-format cameras with 16M or 20M pixels each. The instrument detects sequencing events during a time window of 10 to 100 milliseconds and reports changes that occur within an aperture of about 10 adjacent pixels (16-bits per pixel). The analytes are pipetted with a liquid-handling device into a 96-, 384-, or 1536-well plate and a stage is used to scan each well one at a time. The instrument detects at least 300 bases per minute. The platform can be capable of sequencing a whole human genome (WHG) at standard 30× depth of coverage in three hours. The reduced reagent quantities and efficiencies from analyzing only significant data results in reduced sequencing costs and improvement in speed.

### Example 3: Data Storage

In a particular use of the invention, the regions of the target represent positions of data, whereby the target can be used for data storage and retrieval. Present-day storage solutions are not stable in the long term, and needs to be transferred periodically to new media if it is to be preserved for future generations. The data density of current solid-state media can reach an upper limit of 103 Gb/mm³ whereas the theoretical data density of DNA is 457×10⁹ Gb/g. DNA is readily replicated in multiple copies and can be stable for over 300,000 years. The known issues for DNA storage of latency and readout cost (as well as the synthesis cost) can be overcome with the present invention's inexpensive real-time readout.

The headings provided above are intended only to facilitate navigation within the document and should not be used to characterize the meaning of one portion of text compared to another. Skilled artisans will appreciate that additional embodiments are within the scope of the invention. The invention is defined only by the following claims; limitations from the specification or its examples should not be imported into the claims.

We claim:

1. A method for using a temporal contrast pixel array (TCPA) to detect a change in the emission of an indicator molecule that is bound to a target molecule of interest, wherein the indicator comprises detectable moiety and a binding moiety that is capable of binding specifically to a region of the target.

2. The method of claim 1, wherein the change is selected from the group consisting of a change in intensity, polarization, a rate of increase, duration, decay half-life, decay profile, and emission wavelength of a signal, or a difference in the first, second, or higher order derivative of said changes.

3. The method of claim 2, wherein the change is detected due to a change of the detectable moiety relative to an evanescent field.

4. The method of claim 1, wherein the target is a nucleic acid.

5. The method of claim 1, wherein step (b) is performed on a plurality of targets asynchronously.

6. The method of claim 1, wherein the detection is in real time.

7. The method of claim 1, wherein the detection is performed as a continuous flow of indicators.

8. The method of claim 1, wherein the target comprises a plurality of regions, and the regions represent positions of data, whereby the target can be used for data storage and retrieval.

9. The method of claim 1, whereby the binding constant of two molecules is determined, wherein one of the molecules is selected from the group consisting of a nucleotide, a polynucleotide, an antibody, a Fab, an aptamer, a fatty acid, a lipid, a carbohydrate sugar, a lectin, and a hormone, or a derivative or an analog thereof.

10. A method for detecting the presence of a target molecule of interest in a sample, comprising the steps of:
(a) providing the sample to a chamber;
(b) performing the method of claim 1 on the sample by providing at least a first indicator by
(1) allowing the indicator to bind specifically to a first region of a target;
(2) exciting the indicator to emit a detectable signal; and
(3) reporting a change that is detected in a pixel or group of pixels within an array of the TCPA; and
(c) analyzing the change information to detect the target;
thereby detecting the presence of the target in the sample.

11. A method for detecting the presence of a target molecule of interest having a plurality of regions in a sample, comprising the steps of:
(a) providing the sample to a chamber;
(b) performing the method of claim 1 on the sample by providing at least a first indicator by
(1) allowing the indicator to bind specifically to a first region of a target;
and providing at least a second indicator that can bind to the first region but having a different binding moiety and a different detectable moiety;
(2) exciting the indicator to emit a detectable signal; and
(3) reporting a change that is detected in a pixel or group of pixels within an array of the TCPA;
(4) removing the detectable moiety while continuing to block another binding moiety from binding to the same region of the target;
(c) performing step (b) with a different region of a target; and
(d) analyzing the change information to detect the target.

12. A method for sequencing a nucleic acid in real time, comprising performing the method of claim 11 on unamplified target nucleic acids to detect a nucleotide sequence.

13. The method of claim 12, wherein the detected changes further indicate primary sequence features, methylation or other epigenetic modifications, or secondary structures.

14. A kit comprising a polymerase and at least two indicator molecules,
wherein each indicator comprises a detectable moiety and a binding moiety that is capable of binding specifically to a region of a target molecule of interest, and
wherein the indicators differ by emission wavelength, intensity, duration, or decay profile.

15. The kit of claim 14, further comprising a component selected from the group consisting of a DNA-binding protein, a means for removing the detectable moiety of an indicator, a quenching reagent, a molecular crowding agent, a reagent to reduce or enhance blinking, and a component to increase viscosity, and a sample-chamber flushing fluid.

16. An apparatus comprising:
a TCPA;
fluidic means for delivering an indicator cocktail or a wash solution;
means for communicating changes in image;
a system for collecting and analyzing change data; and
optionally further comprising a component selected from the group consisting of a sample chamber capable of containing a single or multiple samples, an excitation light source, means for controlling the temperature of the sample or the cocktail, an automated liquid handler, and a LIMS system.

17. A device, comprising the apparatus of claim 16 and a sample chamber,
wherein the TCPA optionally comprises a wave-guide surface;
wherein the fluidic means comprises fluidic ports for receiving a sample and optionally an indicator cocktail or a wash solution to the sample chamber; and
wherein the communicating means comprises at least one port for a purpose selected from the group consisting of communicating changes in an image from the TCPA to a system for data collection and
analysis; supplying power to the TCPA; and providing power to the excitation light source.

18. A point-of-care device for performing the method of claim 11 and displaying the results on a mobile electronic device.

19. A computer-performable algorithm for performing the analysis of step (c) of the method of claim 10.

20. A computer-performable method for using an artificial intelligence system to sequence a target nucleic acid, comprising the steps of
(a) training the system with target nucleic acids of known sequence, primary sequence features, methylation or other epigenetic modifications, or secondary structures in order to obtain a model; and
(b) performing the method of claim 12 on a target nucleic acid, wherein the detected changes are characterized by the training model obtained in step (a).
